(19) United States
(12) Patent Application Publication
(10) Pub. No.: US 2017/0042950 A1
(43) Pub. Date: Feb. 16, 2017

(54) MATERIALS AND METHODS FOR IMPROVING GASTROINTESTINAL HEALTH

(71) Applicant: SHAKLEE CORPORATION, Pleasanton, CA (US)

(72) Inventors: Francis C. Lau, Pebble Beach, CA (US); Bruce P. Daggy, Pleasanton, CA (US); Emanuel P. Fakoukakis, Danville, CA (US)

(21) Appl. No.: 15/233,204

(22) Filed: Aug. 10, 2016

Related U.S. Application Data
(60) Provisional application No. 62/203,566, filed on Aug. 11, 2015.

(51) Int. Cl. A61K 35/744; A23L 33/22; A23L 33/21; A23L 33/135

(52) U.S. Cl. CPC A61K 35/744; A23L 33/135; A23L 33/22; A23L 33/21; A61K 2035/11

(57) ABSTRACT

Described herein is a composition comprising a mixture of a probiotic composition and a prebiotic composition, wherein the probiotic composition comprises the bacterium Bacillus coagulans and wherein the prebiotic composition comprises a plant-based fiber, a resistant starch and a short-chain oligosaccharide. Uses of the compositions described herein for maintaining or improving gastrointestinal health is also contemplated.

### CROSS-REFERENCE TO RELATED APPLICATIONS

This application claims the benefit of U.S. Provisional Application No. 62/203,566, entitled “Materials and Methods for Improving Gastrointestinal Health”, filed Aug. 11, 2015, the disclosure of which is hereby incorporated herein by reference in its entirety.

### BACKGROUND OF THE INVENTION

Probiotic microorganisms are generally understood to be microorganisms which beneficially affect a host by improving the host's intestinal microbial balance. In general, it is believed that probiotic organisms produce organic acids such as lactic acid and acetic acid which inhibit the growth of pathogenic bacteria such as Clostridium perfringens and Helicobacter pylori. Probiotic bacteria are therefore believed to be useful in the treatment and prevention of conditions caused by pathogenic bacteria, or an imbalance in intestinal microorganisms. It is also believed that probiotic microorganisms inhibit the growth and activity of putrefying bacteria and hence the production of toxic amine substances, and that they activate the immune function of the host.

Probiotic bacteria have been administered to mammals for years in such foods as fermented dairy products such as yogurts and inoculated pasteurized refrigerated fluid milk. In addition to yogurts, there are available infant and follow-up formulas which contain probiotic microorganisms, such as BIO NAN® formula (available from Societe de Produits Nestle SA). In addition, various products including cereals and nutrition bars that contain probiotic microorganisms have recently become available.

The Bacillus species, particularly those species having the ability to form spores (e.g., Bacillus coagulans), are a preferred embodiment of the present invention. The ability to sporulate makes these bacterial species relatively resistant to heat and other conditions, provides for a long shelf-life in product formulations, and is deal for survival and colonization of tissues under conditions of pH, salinity, and the like within the gastrointestinal tract. Moreover, additional useful properties of many Bacillus species include being non-pathogenic, aerobic, facultative and heterotrophic, thus rendering these bacterial species safe and able to readily colonize the gastrointestinal tract.

The Gram positive rods of Bacillus coagulans have a cell diameter of greater than 1.0 μm with variable swelling of the sporangium, without parasporal crystal production. Bacillus coagulans is a non-pathogenic, Gram positive, spore-forming bacteria that produces L(+) lactic acid (dextrorotatory) under homo-fermentation conditions. It has been isolated from natural sources, such as heat-treated soil samples inoculated into nutrient medium (see e.g., Bergey's Manual of Systemic Bacteriology, Vol. 2, Sneath, P. H. A. et al., eds., Williams & Wilkins, Baltimore, Md., 1986). Purified Bacillus coagulans strains have served as a source of enzymes including endonucleases (e.g., U.S. Pat. No. 5,200,336); amylase (U.S. Pat. No. 4,980,180); lactase (U.S. Pat. No. 4,323,651) and cyclo-malto-dextrin glucano-transferase (U.S. Pat. No. 5,102,800). Bacillus coagulans has also been utilized to produce lactic acid (U.S. Pat. No. 5,079,164). A strain of Bacillus coagulans (also referred to as Lactobacillus sporogenes; Sakaguti & Nakayama, ATCC No. 31284) has been combined with other lactic acid producing bacteria and Bacillus natto to produce a fermented food product from steamed soybeans (U.S. Pat. No. 4,110,477). Bacillus coagulans GBI-30 strain (ATCC Designation Number PTA-6086) has been previously described in U.S. Pat. Nos. 7,713,726 and 8,277,799. Bacillus coagulans strains have also been used as animal feeds additives for poultry and livestock to reduce disease and improve feed utilization and, therefore, to increase growth rate in the animals (International PCT Pat. Applications No. WO 93/14187 and No. WO 94/11492). In particular, Bacillus coagulans strains have been used as general nutritional supplements and agents to control constipation and diarrhea in humans and animals.

Poor gastrointestinal health results from various causes that may produce diarrhea, poor stool quality, or other symptoms. Further, animals must efficiently and properly digest food to maintain good gastrointestinal health. However, poor gastrointestinal health interferes with the ordinary food digestion and adversely affects an animal's health and wellness.

Nutrition research conducted in the 1970's showed that different carbohydrates did not have the same effects on blood glucose (sugar) levels after eating. These findings challenged the general assumption that all ‘complex’ carbohydrates (starches) produce lower blood glucose responses than ‘simple’ sugars, and questioned the clinical significance of carbohydrate exchange lists that have regulated the diets of people with diabetes for over three decades. These exchange lists are based on the assumption that portions of different foods containing equal amounts of carbohydrate will produce the same blood glucose response.

Consequently, the glycemic index (GI) was developed in order to rank equal carbohydrate portions of different foods according to the extent to which they increase blood glucose levels after being eaten. Foods with a high GI value contain rapidly digested carbohydrate, which produces a rapid and large rise and fall in the level of blood glucose. In contrast, foods with a low GI value contain slowly digested carbohydrate, which produces a gradual, relatively low rise in the level of blood glucose and thus control the postprandial glycemic response.

Over two decades of research has confirmed that a food's glycemic effect cannot be accurately predicted from the type and amount of carbohydrate it contains. This is because the rate at which carbohydrate is digested and released into the bloodstream is influenced by many factors, such as the food's physical form, its fat, protein and fiber content, and the chemical structure of its carbohydrate. For these reasons, apparently similar foods within the same food group and different flavors of the same food can have quite different effects on blood glucose levels.

GI research has important implications for the food industry and people's health. Scientists now agree that the terms ‘complex carbohydrate’ and ‘sugars’, which commonly appear on food labels, have little nutritional or physiological significance. The World Health Organization recently released a consensus report stating that these terms should be removed from food labels and replaced with the food's total digestible carbohydrate content and its GI value, in order to help people select foods that will reduce the overall glycemic impact of their diet. Currently, many dietitians refer to the glycemic index when planning more flexible diets for people with diabetes. In addition, GI values are being used in scientific research studies to examine the relationship between the overall glycemic effect of people's habitual diets and their risk of developing certain diseases over time. Results from large-scale epidemiological studies have shown that the long-term consumption of a diet with a high glycemic impact, which induces high and recurrent surges in blood glucose and insulin levels, increases the risk of developing diabetes, heart disease and certain cancers. In contrast, results from both epidemiological and experimental studies show that low-GI diets can reduce the risk of these diseases, improve blood glucose control and insulin sensitivity in people with diabetes, reduce high blood fat levels, and can be useful for weight control. Recently, high-GI diets have been shown to enhance body fat storage to a greater extent than equal-calorie low-GI diets in healthy people, which is likely to reflect the greater insulin secretion and lower satiety associated with high-GI foods. Type 2 diabetes and coronary heart disease continue to be the major causes of illness and death in industrialized countries.

A GI value of 100 represents the reference, an equivalent amount of pure glucose. Foods with a GI value of less than 55 are considered to be low-GI foods. Foods with a GI value between 56-69 are medium- or moderate-GI food and foods with a GI value of 70 or more are high-GI foods.

High-GI diets are not recommended for people with problems in glucose regulation. In addition, high-GI diets may lead to a number of health concerns.

Current methods for maintaining and improving gastrointestinal health often involve modifying the diet, administering various foods thought to effect gastrointestinal health, or administering various drugs thought to be useful for maintaining or improving gastrointestinal health. While those methods have proven useful there still remains a need in the art for more effective methods for maintaining and improving gastrointestinal health.

There also remains a desire in the art for more low-GI foods to assist with the prevention and treatment of diseases associated with high blood sugar.

### BRIEF SUMMARY OF THE INVENTION

The present disclosure is directed to a blend of a probiotic composition and a prebiotic composition for maintaining or improving the gastrointestinal health of a mammalian subject. The disclosure also provides methods and materials for controlling the postprandial glycemic response of humans and other mammals by consumption of the blend of probiotic and prebiotic compositions.

According to one aspect of the invention a composition is provided comprising a mixture of a probiotic composition and a prebiotic composition, wherein the probiotic composition comprises the bacterium Bacillus coagulans and wherein the prebiotic composition comprises a plant-based fiber, a resistant starch and a short-chain oligosaccharide. In some embodiments, the bacterium is the strain of Bacillus coagulans GBI-30 (ATTC Designation No. PTA-6086). The plant-based fiber in the prebiotic composition can be any plant-based fiber known in the art. In some embodiments, the plant-based fiber is selected from the group consisting of plant fiber selected from the group consisting of fiber from soy, sacha inchi, pea, potato, rice, konjac, oats, guar gum, pectin and psyllium. Similarly, the resistant starch in the prebiotic composition can be any resistant starch known in the art. In some embodiments, the resistant starch is a sugar-free digestion-resistant dextrin with an average degree of polymerization of 18. In some embodiments, the resistant starch is selected from the group consisting of resistant dextrin from corn. Likewise, the short-chain oligosaccharide can be any short-chain oligosaccharide known in the art. In some embodiments, the short-chain oligosaccharide is selected from the group consisting of a fructooligosaccharide (FOS), a galactooligosaccharide (GOS), and oligopolymers of fructose, glucose, galactose, or combinations thereof. In some embodiments, the short-chain oligosaccharide is a FOS, such as a FOS comprising β2-1 linked linear chains of fructose bound to a terminal glucose and having the degree of polymerization ranging from 3 to 5. In some embodiments, the FOS is selected from the group consisting of kestose (glucose-fructose-fructose or GF₂), nystose (GF₃), fructosyl-nystose (GF₄), GF₅, or GF_{5+m (where m=1 to 100)}.

The composition comprising the mixture of the probiotic composition and the prebiotic composition disclosed herein can be in any form suitable for administering the composition to a mammalian subject. In some embodiments, the composition is in the form of a tablet, a powder or a liquid. If provided as a powder, combining the powder with a suitable liquid (e.g., liquid dairy product, fruit or vegetable juice, blended fruit or vegetable juice product, etc.) is specifically contemplated.

In another aspect, disclosed herein is a powdered formulation comprising the strain of Bacillus coagulans GBI-30 (ATTC Designation No. PTA-6086) in an amount ranging from 1×10⁶ CFUs to 1×10¹⁴ CFUs, a resistant dextrin in an amount ranging from 0.1 g to about 25 g, a plant-based fiber in an amount ranging from 0.1 g to about 25 g, and a fructooligosaccharide in an amount ranging from 0.1 g to about 25 g.

In another aspect, disclosed herein is a powdered formulation comprising the strain of Bacillus coagulans GBI-30 (ATTC Designation No. PTA-6086) in an amount of about 1×10⁹ CFUs, a resistant dextrin in an amount of about 3.7 g, a plant-based fiber in an amount of about 1.3 g, and a fructooligosaccharide in an amount ranging from 1.2 g. In some embodiments, the powdered formulation further comprises insulin.

In another aspect, disclosed herein is a powdered formulation comprising the strain of Bacillus coagulans GBI-30 (ATTC Designation No. PTA-6086) in an amount of about 1×10⁹ CFUs, a resistant dextrin in an amount of about 5 g, and a fructooligosaccharide in an amount ranging from 1.2 g. In some embodiments, the powdered formulation further comprises insulin.

In another aspect, disclosed here is a method of maintaining or improving gastrointestinal health in a mammalian subject comprising administering to the subject a composition comprising a mixture of a probiotic composition and a prebiotic composition, wherein the probiotic composition comprises the bacterium Bacillus coagulans and wherein the prebiotic composition comprises a plant-based fiber, a resistant starch and a short-chain oligosaccharide. In some embodiments, the mammalian subject is a human.

The compositions can be consumed in the form of a tablet, a powder or a liquid and can be consumed separately or with other foods or beverages.

This application incorporates by reference the disclosure of co-owned and copending U.S. Ser. No. ______ filed Aug. 10, 2016 (Docket No. 32550/49446A) which claims benefit of Provisional Application Ser. No. 62/203,564 filed Aug. 11, 2015 the disclosure of which is also incorporated by reference.

### BRIEF DESCRIPTION OF THE DRAWING

FIG. 1 depicts the average plasma glucose response curves for the equal-carbohydrate portions of the reference food and three protein products containing a probiotic and prebiotic composition, shown as the change in plasma glucose from the fasting baseline level.

### DETAILED DESCRIPTION OF THE INVENTION

The present disclosure is directed to a blend of a probiotic composition and a prebiotic composition for maintaining or improving the gastrointestinal health of a mammalian subject. According to one aspect of the invention described herein a composition is provided comprising a mixture of a probiotic composition and a prebiotic composition, wherein the probiotic composition comprises the bacterium Bacillus coagulans and wherein the prebiotic composition comprises a plant-based fiber, a resistant starch and a short-chain oligosaccharide.

Probiotic Composition

As used herein, the term, “probiotic” refers to microorganisms that form at least a part of the transient or endogenous flora and thereby exhibit a beneficial prophylactic and/or therapeutic effect on the host organism. Probiotics are generally known to be clinically safe (i.e., non-pathogenic) by those individuals skilled in the art. By way of example, and not of limitation to any particular mechanism, the prophylactic and/or therapeutic effect of an acid-producing bacteria of the present invention results, in part, from a competitive inhibition of the growth of pathogens due to: (i) their superior colonization abilities; (ii) parasitism of undesirable microorganisms; (iii) the production of acid (e.g., lactic, acetic, and other acidic compounds) and/or other extracellular products possessing anti-microbial activity; and (iv) various combinations thereof. It should be noted that the aforementioned products and activities of the acid-producing bacteria of the present invention act synergistically to produce the beneficial probiotic effect disclosed herein.

Bacillus coagulans cultures have been deposited with the following primary international culture collections: Agricultural Research Service Culture Collection; Russian Collection of Microorganisms; Deutsche Sammlung von Mikroorganismen und Zellkulturen GmbH (German Collection of Microorganisms and Cell Cultures, VKM DSMZ); American Type Culture Collection (ATCC); Finnish Microorganism Collection (University of Goteborg, Sweden); Japan Collection of Microorganisms (JCM); and Japan Federation for Culture Collection.

Various Bacillus coagulans bacterial strains which are currently commercially available from the American Type Culture Collection (ATCC, Rockville, Md.) include the following accession numbers: Bacillus coagulans Hammer NRS 727 (ATCC No. 11014); Bacillus coagulans Hammer strain C (ATCC No. 11369); Bacillus coagulans Hammer (ATCC No. 31284); and Bacillus coagulans Hammer NCA 4259 (ATCC No. 15949). Purified Bacillus coagulans bacteria are also available from the Deutsche Sarumlung von Mikroorganismen und Zellkuturen GmbH (Braunschweig, Germany) using the following accession numbers: Bacillus coagulans Hammer 1915 (DSM No. 2356); Bacillus coagulans Hammer 1915 (DSM No. 2383, corresponds to ATCC No. 11014); Bacillus coagulans Hammer (DSM No. 2384, corresponds to ATCC No. 11369); and Bacillus coagulans Hammer (DSM No. 2385, corresponds to ATCC No. 15949). Bacillus coagulans bacteria can also be obtained from commercial suppliers such as Sabinsa Corporation (Piscataway, N.J.) or K.K. Fermentation (Kyoto, Japan).

These aforementioned Bacillus coagulans strains and their growth requirements have been described previously (see e.g., Baker, D. et al, 1960. Can. J. Microbiol. 6: 557-563; Nakamura, H. et al, 1988. Int. J. Svst. Bacteriol. 38: 63-73. In addition, various strains of Bacillus coagulans can also be isolated from natural sources (e.g., heat-treated soil samples) using well-known procedures (see e.g., Bergey's Manual of Systemic Bacteriology, Vol. 2, p. 1117, Sneath, P. H. A. et al., eds., Williams & Wilkins, Baltimore, Md., 1986).

Bacillus coagulans had originally been mis-characterized as a Lactobacillus in view of the fact that, as originally described, this bacterium was labeled as Lactobacillus sporogenes (See, Nakamura et al. 1988. Int. J. Syst. Bacteriol. 38: 63-73). However, initial classification was incorrect due to the fact that Bacillus coagulans produces spores and through metabolism excretes L(+)-lactic acid, both aspects which provide key features to its utility. Instead, these developmental and metabolic aspects required that the bacterium be classified as a lactic acid Bacillus, and therefore it was re-designated.

In some embodiments, the Bacillus coagulans provided in the compositions and used in the methods described herein is Bacillus coagulans GBI-30 strain (ATCC Designation Number PTA-6086) as described in U.S. Pat. Nos. 7,713,726 and 8,277,799, the disclosures of which are incorporated herein by reference in their entireties.

In some embodiments, the probiotic composition described herein comprises Bacillus coagulans GBI-30 strain (ATCC Designation Number PTA-6086) in an amount ranging from 1×10³ to about 1×10¹⁴ CFU (e.g., 1×10³, 1×10⁴, 1×10⁵, 1×10⁶, 1×10⁷, 1×10⁸, 1×10⁹, 1×10¹⁰, 1×10¹¹, 1×10¹², 1×101³, or 1×10¹⁴) of viable, vegetative bacteria or spore. In some embodiments, the probiotic composition described herein comprises Bacillus coagulans GBI-30 strain (ATCC Designation Number PTA-6086) in an amount of about 1×10⁹ CFU of viable, vegetative bacteria or spore.

Prebiotic Composition

The composition described herein comprises a prebiotic composition in addition to the probiotic a composition. A “prebiotic” is a non-digestible substance (or substances) that when consumed provide a beneficial physiological effect on the host by selectively stimulating the favorable growth or activity of a limited number of bacteria (Gibson G R, Roberfroid M B. Dietary modulation of the human colonic microbiota: introducing the concept of prebiotics. J Nutr. 1995 June; 125(6): 1401-12). A prebiotic is generally a saccharide that is non-digestible or essentially non-digestible by a human or another mammal and acts to encourage the growth of probiotic bacteria in the gut, increase adhesion of probiotic bacteria in the gut, displace pathogens, or provide a fermentable dose of carbohydrate to probiotic bacteria (symbiotic) or selected commensal bacteria and increase the levels of those microbial populations (notably lactobacilli, Bacillus coagulans and bifidobacteria) in the gastrointestinal tract. A prebiotic can be a saccharide that is non-digestible by a human or other mammalian host and can act as a non-digestible fiber in the diet. This non-digestibility is because humans lack the enzymes to break down some or all of the prebiotic oligosaccharide as it travels through the digestive tract. When a prebiotic reaches the small intestine and colon, bacteria producing an enzyme or enzymes capable of digesting the prebiotic can break down the prebiotic into simple sugars that the bacteria can use.

Suitable prebiotics can include one or more of a carbohydrate, carbohydrate monomer, carbohydrate oligomer, or carbohydrate polymer. In one embodiment, the prebiotics are non-digestible saccharides, which include non-digestible monosaccharides, non-digestible oligosaccharides, or non-digestible polysaccharides. In one embodiment, the sugar units of an oligosaccharide or polysaccharide can be linked in a single straight chain or can be a chain with one or more side branches. The length of the oligosaccharide or polysaccharide can vary from source to source. In one embodiment, small amounts of glucose can also be contained in the chain. In another embodiment, the prebiotic composition can be partially hydrolyzed or contain individual sugar moieties that are components of the primary oligosaccharide.

In some embodiments, the prebiotic composition comprises a plant-based fiber, a resistant starch and a short-chain oligosaccharide. The plant-based fiber in the prebiotic composition can be any plant-based fiber known in the art. In some embodiments, the plant-based fiber selected from the group consisting of fiber from soy, sacha inchi, pea, potato, rice, konjac, oats, guar gum, pectin and psyllium.

Similarly, the resistant starch in the prebiotic composition can be any resistant starch known in the art. The term “resistant starch” as used herein refers to the sum of starch and products of starch digestion not absorbed in the small intestine of healthy humans but entering into the large bowel. This is defined in terms of a percentage of the total starch of the grain, or a percentage of the total starch content in the food, according to the context. Thus, resistant starch excludes products digested and absorbed in the small intestine. Resistant starches include physically inaccessible starch (RS1 form), resistant native starch granules (RS2), retrograded starches (RS3), and chemically modified starches (RS4). In some embodiments, the resistant starch is a sugar-free digestion-resistant dextrin with an average degree of polymerization of 18. In some embodiments, the resistant starch is selected from the group consisting of resistant dextrin derived from wheat corn or other plant sources. According to one preferred aspect of the invention the resistant dextrin is produced from maize such as commercially available as FM06 from Roquette, Keokuk, Iowa.

Likewise, the short-chain oligosaccharide wherein the short-chain oligosaccharide is selected from the group consisting of a fructooligosaccharide (FOS), a galactooligosaccharide (GOS), or oligopolymers of fructose, glucose, galactose or other sugar molecules alone, or combinations thereof. In some embodiments, the short-chain oligosaccharide is a FOS, such as a FOS comprising β2-1 linked linear chains of fructose bound to a terminal glucose and having the degree of polymerization ranging from 3 to 5. In some embodiments, the FOS is selected from the group consisting of kestose (glucose-fructose-fructose or GF₂), nystose (GF₃), fructosyl-nystose (GF₄), GF₅, or GF_{5+m (where m=1 to 100)}. According to one aspect of the invention the short chain oligosaccharide is a β2-1 linked liner chain of fructose bound to a terminal glucose commercially available as Nutraflora® available from Ingredion, Westchester, Ill.

In another aspect, disclosed herein is a powdered formulation comprising the strain of Bacillus coagulans GBI-30 (ATTC Designation No. PTA-6086) in an amount ranging from 1×10⁶ CFUs to 1×10¹⁴ CFUs, a resistant dextrin in an amount ranging from 0.1 g to about 25 g, a plant-based fiber in an amount ranging from 0.1 g to about 25 g, and a fructooligosaccharide in an amount ranging from 0.1 g to about 25 g.

In another aspect, disclosed herein is a powdered formulation comprising the strain of Bacillus coagulans GBI-30 (ATTC Designation No. PTA-6086) in an amount of about 1×10⁹ CFUs, a resistant dextrin in an amount of about 3.7 g, a plant-based fiber in an amount of about 1.3 g, and a fructooligosaccharide in an amount ranging from 1.2 g. In some embodiments, the powdered formulation further comprises insulin.

In another aspect, disclosed herein is a powdered formulation comprising the strain of Bacillus coagulans GBI-30 (ATTC Designation No. PTA-6086) in an amount of about 1×10⁹ CFUs, a resistant dextrin in an amount of about 5 g, and a fructooligosaccharide in an amount ranging from 1.2 g. In some embodiments, the powdered formulation further comprises insulin.

Routes of Administration

Administration of a composition described herein to the gastrointestinal tract using a gel, suspension, aerosol spray, capsule, tablet, powder or semi-solid formulation (e.g., a suppository) is specifically contemplated. Administration of the compositions containing the active probiotic lactic acid-producing bacterium which is effective in preventing or treating a pathogenic bacterial infection, generally consist of one to ten dosages of approximately 10 mg to 10 g of the therapeutic composition per dosage, for a time period ranging from one day to one month. Administrations are (generally) once every twelve hours and up to once every four hours. In the preferred embodiment, two to four administrations of the therapeutic composition per day, of approximately 0.1 g to 5 g per dose, for one to seven days.

In some embodiments the composition comprising the prebiotic composition and the probiotic composition described herein is incorporated into a food product. The term “food product” as used herein refers to any substance containing nutrients that can be ingested by an organism to produce energy, promote health and wellness, stimulate growth, and maintain life. The term “enriched food product” as used herein refers to a food product that has been modified to include the composition comprising the prebiotic composition and the probiotic composition described herein, which provides a benefit such as a health/wellness-promoting and/or disease-preventing/mitigating/treating property beyond the basic function of supplying nutrients.

The composition comprising the prebiotic composition and the probiotic composition can be incorporated into any food product. Exemplary food products include, but are not limited to, protein powder (meal shakes), baked goods (cakes, cookies, crackers, breads, scones and muffins), dairy-type products (including but not limited to cheese, yogurt, custards, rice pudding, mousses, ice cream, frozen yogurt, frozen custard), desserts (including, but not limited to, sherbet, sorbet, water-ices, granitas and frozen fruit purees), spreads/margarines, pasta products and other cereal products, meal replacement products, nutrition bars, trail mix, granola, beverages (including, but not limited to, smoothies, water or dairy beverages and soy-based beverages), and breakfast type cereal products such as oatmeal. For beverages, the composition comprising the prebiotic composition and the probiotic composition described herein may be in solution, suspended, emulsified or present as a solid.

In one embodiment, the enriched food product is a meal replacement product. The term “meal replacement product” as used herein refers to an enriched food product that is intended to be eaten in place of a normal meal. Nutrition bars and beverages that are intended to constitute a meal replacement are types of meal replacement products. The term also includes products which are eaten as part of a meal replacement weight loss or weight control plan, for example snack products which are not intended to replace a whole meal by themselves, but which may be used with other such products to replace a meal or which are otherwise intended to be used in the plan. These latter products typically have a calorie content in the range of from 50-500 kilocalories per serving.

In another embodiment, the food product is a dietary supplement. The term “dietary supplement” as used herein refers to a substance taken by mouth that contains a “dietary ingredient” intended to supplement the diet. The term “dietary ingredients” includes, but is not limited to, the composition comprising the prebiotic composition and the probiotic composition as described herein as well as vitamins, minerals, herbs or other botanicals, amino acids, and substances such as enzymes, organ tissues, glandulars, and metabolites.

In yet another embodiment, the food product is a medical food. The term “medical food” as used herein means a food which is formulated to be consumed or administered entirely under the supervision of a physician and which is intended for the specific dietary management of a disease or condition for which distinctive nutritional requirements, based on recognized scientific principles, are established by medical evaluation.

In yet another embodiment, the composition comprising the probiotic composition and the prebiotic composition described herein are incorporated into a pharmaceutical product or composition. Pharmaceutical compositions comprise a prophylactically or therapeutically effective amount of the composition described herein and typically one or more pharmaceutically acceptable carriers or excipients (which are discussed below).

The disclosure contemplates compositions comprising the probiotic composition and the prebiotic composition described herein that are, in some embodiments, powdered, tabletted, encapsulated or otherwise formulated for oral administration. The compositions may be provided as pharmaceutical compositions, nutraceutical compositions (e.g., a dietary supplement), or as a food or beverage additive, as defined by the U.S. Food and Drug Administration. The dosage form for the above compositions are not particularly restricted. For example, liquid solutions, suspensions, emulsions, tablets, pills, capsules, sustained release formulations, powders, suppositories, liposomes, microparticles, microcapsules, sterile isotonic aqueous buffer solutions, and the like are all contemplated as suitable dosage forms.

The compositions typically include one or more suitable diluents, fillers, salts, disintegrants, binders, lubricants, glidants, wetting agents, controlled release matrices, colorings, flavoring, carriers, excipients, buffers, stabilizers, solubilizers, commercial adjuvants, and/or other additives known in the art.

Any pharmaceutically acceptable (i.e., sterile and acceptably non-toxic as known in the art) liquid, semisolid, or solid diluent that serves as a pharmaceutical vehicle, excipient, or medium can be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch.

Pharmaceutically acceptable fillers can include, for example, lactose, microcrystalline cellulose, dicalcium phosphate, tricalcium phosphate, calcium sulfate, dextrose, mannitol, and/or sucrose. Salts, including calcium triphosphate, magnesium carbonate, and sodium chloride, may also be used as fillers in the pharmaceutical compositions.

Binders may be used to hold the composition together to form a hard tablet. Exemplary binders include materials from organic products such as acacia, tragacanth, starch and gelatin. Other suitable binders include methyl cellulose (MC), ethyl cellulose (EC) and carboxymethyl cellulose (CMC).

In some embodiments, an enriched food product further comprises a bioavailability enhancer, which acts to increase the absorption of the sorbable natural product(s) by the body. Bioavailability enhancers can be natural or synthetic compounds. In one embodiment, the enriched food product comprising the composition described herein further comprises one or more bioavailability enhancers in order to enhance the bioavailability of the bioactive natural product(s).

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.). In some embodiments, the natural bioavailability enhancers is present in an amount of from about 0.02% to about 0.6% by weight based on the total weight of enriched food product.

Examples of suitable synthetic bioavailability enhancers include, but are not limited to surfactants including those composed of PEG-esters such as are commercially available under the tradenames: Gelucire®, Labrafil® and Labrasol®, Lauroglycol®, Pleurol Oleique® (Gattefosse Corp., Paramus, N.J.) and Capmul® (Abitec Corp., Columbus, Ohio).

The amount and administration regimen of the composition is based on various factors relevant to the purpose of administration, for example human or animal age, sex, body weight, hormone levels, or other nutritional need of the human or animal. In some embodiments, the composition is administered to a mammalian subject in an amount from about 0.001 mg/kg body weight to about 1 g/kg body weight.

A typical regimen may comprise multiple doses of the composition. In one embodiment, the composition is administered once per day. The composition may be administered to an individual at any time. In some embodiments, the composition is administered concurrently, or prior to or at the consumption of a meal.

All of the U.S. patents, U.S. patent application publications, U.S. patent applications, foreign patents, foreign patent applications and non-patent publications referred to in this specification and/or listed in the Application Data Sheet, are incorporated herein by reference, in their entireties.

From the foregoing it will be appreciated that, although specific embodiments of the invention have been described herein for purposes of illustration, various modifications may be made without deviating from the spirit and scope of the invention.

### EXAMPLE 1

### Assessment of Survival and Metabolic Activity of Bacillus coagulans GBI-30 (ATTC Designation No. PTA-6086) in an In Vitro Model of the Stomach and Small Intestine

A digestion experiment to assess the survival and metabolic activity of Bacillus coagulans is carried out as described in Maathuis et al., Beneficial Microbes, 1:31-36, 2010 wherein the survivability and metabolic activity of Bacillus coagulans) GBI-30 (ATTC Designation No. PTA-6086) when combined with the prebiotic composition described herein during passage through the upper gastro-intestinal tract is tested in an in vitro model of the stomach and small intestine (the disclosure of which is incorporated herein by reference) in an in vitro model of the stomach and small intestine. The study is performed in the TNO dynamic, multi-compartmental system of the stomach and small intestine (TIM-1) as described in Minekus et al., Appl. Microbiol. and Biotechnol., 53:108-114, 1999, the disclosure of which is incorporated herein by reference. It is contemplated that samples having received both the probiotic and prebiotic compositions described herein will have a higher survival rate than samples having not received the prebiotic composition.

### EXAMPLE 2

### Effect on the Fecal Microbiota of Subjects after Dietary Supplementation with the Prebiotic/Probiotic Blend

The experiments described in Nyangale et al., Anaerobe, 30:75-81, 2014 (the disclosure of which is incorporated by reference in its entirety) are carried out on human volunteers using a composition comprising the probiotic and prebiotic compositions described herein as the test composition (with either the probiotic composition or the prebiotic composition being used as a control. It is contemplated that fecal matter obtained from subjects having received the composition comprising the prebiotic composition and the probiotic composition will have higher populations of the of Bacillus coagulans GBI-30 (ATTC Designation No. PTA-6086) compared to subjects receiving one of the control compositions.

### EXAMPLE 3

### Effect on Glycemic Index of Subjects after Dietary Supplementation with the Prebiotic/Probiotic Blend

According to this example, a prebiotic/probiotic blend according to the invention (Bacillus coagulans and pre-biotic fibers (a plant-based fiber, a resistant starch and a short-chain oligosaccharide) was tested to determine its effect on the glycemic index (GI) when present in a non-soy based nutrient shake comprising a combination of proteins including Sacha inchi protein, pea protein, rice protein and potato protein or in a nutrient shake containing soy protein. The glycemic index is a number associated with a particular type of food that indicates the food's effect on a person's blood glucose (sugar) level. The GI represents the total rise in a person's blood sugar level following consumption of the food.

Testing was carried out using internationally recognized GI methodology (Joint FAO/WHO Report. Carbohydrates in Human Nutrition. FAO Food and Nutrition, Paper 66. Rome: FAO, 1998.), which has been validated by results obtained from small experimental studies and large multi-center research trials (Wolever T M S et al. Determination of the glycemic index values of foods: an interlaboratory study. European Journal of Clinical Nutrition 2003; 57: 475-482). The experimental procedures used in this study were in accordance with international standards for conducting ethical research with humans and were approved by the Medical Ethics Review Committee of Sydney University.

A group of ten healthy, non-smoking people, aged between 18-65 years, was recruited from the staff and student population of the University of Sydney. (A power-based (90%) sample size calculation using data from many published GI studies indicated that a group of at least 10 people would be needed for this study in order to find a significant difference among the GI values of the test foods and the reference food, if a significant difference truly exists (a difference of 1.0 standard deviation units in GI.)) People volunteering to participate in the study were excluded if they: were over- or underweight; were dieting; had impaired glucose tolerance; were suffering from any illness or food allergy; or were regularly taking prescription medication other than standard contraceptive medication. The group that participated in the study consisted of six females and four males. The average age of the subjects was 26.4 years (range: 19.9-34.8 years) and the group's average body mass index (BMI) score was 21.2 kg/m2 (range: 19.4-24.7 kg/m2). The BMI score is a measure of a person's weight in relation to their height. BMI values between 18-25.0 kg/m2 are within the healthy weight range.

Test Foods

Glucose (reference food)

Nutrient Shake, Soy (vanilla)—shaker preparation

Nutrient Shake, Non-Soy (vanilla)—shaker preparation

Nutrient Shake, Non-Soy (vanilla)—blender preparation

According to this example, prebiotic components of a resistant starch dextrin which is partially hydrolyzed maize and is commercially available from Roquette, Keokuk, Iowa as FM06, a short chain oligosaccharide is a β2-1 linked liner chain of fructose bound to a terminal glucose commercially available as Nutraflora® available from Ingredion, Westchester, Ill. and a probiotic comprising at least 1 billion viable CFU of Bacillus coagulans GBI-30 were incorporated into soy and non-soy nutrient shakes which further contained plant-based dietary fiber in the amounts presented in Table 1 below. In the case of the soy nutrient shake the fiber was primarily soy fiber which naturally accompanied the extracted soy protein. In the case of the non-soy shake the fiber was primarily fiber which accompanied pea protein and Sacha inchi protein present in the shake composition. The shakes contained other protein, carbohydrates, fats, vitamins and other nutrients with the calorie counts and other nutritional information set out in Table 2 below. In each case, the amount of fiber totals to 6 grams per serving.

TABLE 1
Fiber Components -6 grams per serving

| | Dextrin | SC FOS | Other Fiber | BC₃₀ |
|---|---|---|---|---|
| Shake | (g) | (g) | (g) | (CFU) |
| Soy Vanilla | 3.0 | 1.2 | 1.8 | 1 billion |
| Non-Soy Vanilla | 4.1 | 1.2 | 0.7 | 1 billion |

TABLE 2
The weights and carbohydrate contents of the test portions of the reference
food and the three shakes, calculated using manufacturers' data.

| | Portion | Energy | Protein | Fat | Available | Sugar | Fiber |
|---|---|---|---|---|---|---|---|
| Test food | Size (g) | (kJ) | (g) | (g) | Carbohydrate (g) | (g) | (g) |
| Reference food | 25.7 g glucose | 400 | 0.0 | 0.0 | 25.0 | 25.0 | 0.0 |
| (glucose sugar) | 250 mL water | | | | | | |
| Soy Life Shake | 89.6 g powder | 1481 | 33.3 | 6.3 | 25.0 | 20.8 | 12.5 |
| | 520.8 mL water | | | | | | |
| Non-Soy Life | 93.8 g powder | 1481 | 33.4 | 6.3 | 25.0 | 20.8 | 12.5 |
| Shake - shaker | 521.1 mL water | | | | | | |
| Non-Soy Life | 93.8 g powder | 1481 | 33.4 | 6.3 | 25.0 | 20.8 | 12.5 |
| Shake - blender | 521.1 mL water | | | | | | |

Each test portion of the nutrient shakes was prepared according to the manufacturer's instructions immediately before required. For the two shakes that were prepared using the shaker method, the appropriate amount of powder and cold water were placed into a plastic shaker container and mixed well by manual shaking for 1 minute until combined. For the shake that was prepared using the blender method, the appropriate amount of powder and cold water were placed into blender and mixed well for 20 seconds until combined. Each prepared shake was served to a subject with 250 mL of plain water. The subjects were required to consume all fluid served.

Using standard methodology to determine a food's GI value, a portion of the food containing between 25 and 50 grams of available carbohydrate was fed to the group of ten healthy people the morning after they have fasted overnight. A fasting blood sample was obtained and then the food was consumed, after which additional blood samples were obtained at regular intervals during the next two hours. In this way, it was possible to measure the total increase in blood sugar (glucose) produced by that food over a two-hour period.

The same procedure was repeated in the same group of people on another day after they have consumed a portion of the reference food (pure glucose sugar in water) containing an equal amount of available carbohydrate. A GI value for the test food can then be calculated by expressing the two-hour blood glucose response to the test food as a percentage of the response produced by the reference food (GI value of glucose=100). Therefore, GI values for foods are relative measures which indicate how high blood sugar levels rise after eating a particular food compared to the very high blood sugar response produced by the same amount of carbohydrate in the form of glucose sugar. Equal-carbohydrate portions of the test foods and reference food are used in GI studies, because carbohydrate is the nutrient in food that directly causes the blood's glucose level to rise.

In this study, the ten healthy people each consumed the reference food on three separate occasions and each of the test foods on one occasion only. Therefore, subjects completed six test sessions. The reference food was consumed on the first, fourth and sixth test sessions, and the test foods were consumed in random order in between. Each session was completed on a separate morning with at least a day in between subsequent sessions.

For each subject, the concentration of glucose in the plasma component of each of the eight plasma samples collected during each two-hour test session was analyzed in duplicates. A two-hour blood glucose response curve was constructed for each subject's reference food and test food sessions using the average plasma glucose concentrations for each of their blood samples. The two fasting blood samples were averaged to provide one baseline glucose concentration.

The incremental area under each two-hour plasma glucose curve (iAUC) was then calculated in order to obtain a single number, which expresses the total increase in plasma glucose in that subject as a result of ingesting that food during the two-hour period. A GI value for each test product was then calculated for each subject by dividing their two-hour blood glucose iAUC values for each test food by their average two-hour blood glucose iAUC value for the reference food and multiplying by 100 to obtain a percentage value (equation 1). Due to differences in body weight and metabolism, blood glucose responses to the same food can vary between different people. The use of the reference food to calculate GI values reduces the variation between the subjects' blood glucose results to the same food arising from these natural differences. Therefore, the GI value for the same food varies less between subjects than their glucose AUC values for this food.

$$\begin{matrix}GI value for test food ( \% ) = \frac{Plasma glucose iAUC value of test food}{\begin{matrix}Average iAUC value of equal \text{-} carbohydrate portion \\ of reference Food\end{matrix}} \times 100\end{matrix} \qquad Equation 1$$

Results And Conclusions

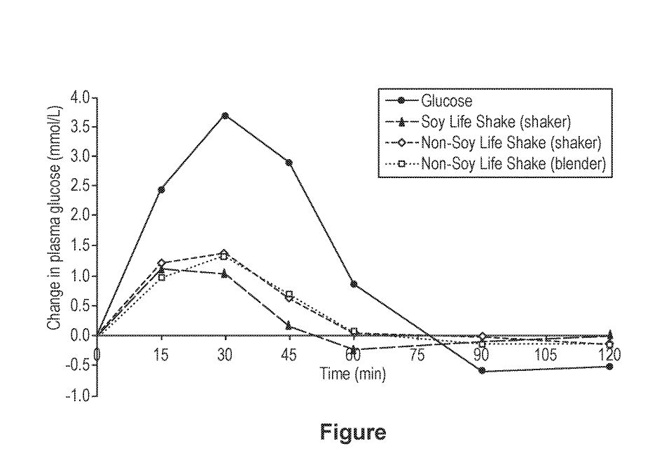

The average two-hour plasma glucose response curves for the 25-gram carbohydrate portions of the reference food and the three prepared Life Shake products are shown in FIG. 1. The reference food was rapidly absorbed, producing a high peak plasma glucose concentration at 30 minutes and the largest overall glycemic response. All three test foods produced substantially lower peak plasma glucose concentrations and overall glycemic responses than the reference food. Different preparation (manual shaking vs. blender mixing) did not affect GI values.

The three shakes prepared with shaker or blender produced average GI values of 26-39, which place these products well within the low GI category (Table 3). Using glucose as the reference food (GI=100), foods with a GI value less than 55 are currently considered to be low-GI foods. Foods with a GI value between 56-69 are medium-GI foods, and foods with a GI value of 70 or more are high-GI foods. Therefore, these shakes would be suitable for consumption in controlled amounts by people with difficulty blood glucose regulation.

TABLE 3
The average ± SEM GI values for the shakes and reference food.

| Test Food | GI Value | GI Category |
|---|---|---|
| Soy Vanilla Life Shake (Shaker) | 26 | Low |
| Non-Soy Vanilla Life Shake (Shaker) | 35 | Low |
| Non-Soy Vanilla Life Shake (Blender) | 39 | Low |
| Reference Food (Glucose) | 100 | High |

GI values are measured using portions of foods and drinks that contain between 25-50 grams of digestible carbohydrate, but these may not be similar to the amounts of these products typically consumed by people in normal environments. It is possible to calculate a glycemic load (GL) value for any sized portion of a carbohydrate-containing food, as long as you know it's GI value. The GL value for a food or drink is calculated according to equation 2 below.

GL value for test foods=(amount of carbohydrate per serving×GI value)/100 Equation 2

Similar to GI values, GL values are useful for helping people identify which types and amounts of foods will produce relatively lower blood glucose responses after consumption—an important consideration for people with diabetes and at risk of developing it. Currently, the consensus is that GL values of 10 or less are low GL; GL values between 11-19 are medium GL values; and GL values of 20 or more are high GL values. The GL values for a standard serve of each of the products tested in this study are listed below:

1. Life Shake Vanilla (Soy) (shaker preparation) (43 g/serving+250 mL water):

(12 g Carb×26 GI)/100=3

2. Life Shake Vanilla (Non-Soy) (shaker preparation) (45 g/serving+250 mL water):

(12 g Carb×39 GI)/100=5

3. Life Shake Vanilla (Non-Soy) (blender preparation) (45 g/serving+250 mL water):

(12 g Carb×35 GI)/100=4

The three shakes tested in this study produced GL values ranging from 3-5, which places these products in the low GL category. It is therefore clear that the shakes containing the compositions of the invention have a reduced glycemic response despite their overall caloric, sugar and total carbohydrate contents.

### EXAMPLE 4

### Effect of Blending on Probiotic Bacteria Survival

According to this example the effect of medium and high speed blending on the survivability of the Bacillus coagulans GBI-30 strain was determined. Specifically, a 43 gram sample of a non-soy vanilla shake sample containing about 1 billion CFU of GBI-30 Bacillus coagulans was mixed with 240 ml of nonfat milk at about 4° C. in a blender at medium and at high speeds with 15 second and 1 minutes blender times as well as mixing in a shaker bottle and shaken by hand. CFU counts were measured after incubation for 7 days at 30° C. with the colonies then counted. No significant difference in counts and thus viability was determined indicating that survivability was not affected by blending.

Numerous modifications and variations in the practice of the invention are expected to occur to those of skill in the art upon consideration of the presently preferred embodiments thereof. Consequently, the only limitations which should be placed upon the scope of the invention are those which appear in the appended claims.

What is claimed:

1. A composition comprising a mixture of a probiotic composition and a prebiotic composition, wherein the probiotic composition comprises the bacterium Bacillus coagulans and wherein the prebiotic composition comprises a plant-based fiber, a resistant starch and a short-chain oligosaccharide.

2. The composition of claim 1, wherein the bacterium is the strain of Bacillus coagulans GBI-30 (ATTC Designation No. PTA-6086).

3. The composition of claim 1, wherein the plant-based fiber is selected from the group consisting of fiber from soy, sacha inchi, pea, potato, rice, konjac, oats, guar gum, pectin and psyllium.

4. The composition of claim 1, wherein the resistant starch is selected from the group consisting of resistant dextrin derived from wheat, corn or other plant sources.

5. The composition of claim 1, wherein the short-chain oligosaccharide is selected from the group consisting of a fructooligosaccharide (FOS), a galactooligosaccharide (GOS), and oligopolymers of fructose, glucose, galactose or other sugar molecules alone or in any combinations

6. The composition of claim 5, wherein the short-chain oligosaccharide is a FOS.

7. The composition of claim 6, wherein the FOS is selected from the group consisting of kestose (glucose-fructose-fructose or GF₂), nystose (GF₃), fructosyl-nystose (GF₄), GF₅, or GF_{5+m (where m=1 to 100)}, and combinations thereof.

8. The composition of claim 1, that is in the form of a tablet, a powder or a liquid.

9. A method of maintaining or improving gastrointestinal health in a mammalian subject comprising administering to the subject a composition comprising a mixture of a probiotic composition and a prebiotic composition, wherein the probiotic composition comprises the bacterium Bacillus coagulans and wherein the prebiotic composition comprises a plant-based fiber, a resistant starch and a short-chain oligosaccharide.

10. The method of claim 10, wherein bacterium is the strain of Bacillus coagulans GBI-30 (ATTC Designation No. PTA-6086).

11. The method of claim 10, wherein the plant-based fiber is selected from the group consisting of fiber from soy, sacha inchi, pea, potato, rice, konjac, oats, guar gum, pectin and psyllium.

12. The method of claim 10, wherein the resistant starch is selected from the group consisting of resistant dextrin derived from wheat, corn or other plant sources.

13. The method of claim 10, wherein the short-chain oligosaccharide is selected from the group consisting of a fructooligosaccharide (FOS), a galactooligosaccharide (GOS), and oligopolymers of fructose, glucose, galactose or other sugar molecules alone or in any combinations

14. The method of claim 13 wherein the short-chain oligosaccharide is a FOS.

15. The method of claim 14, wherein the FOS is selected from the group consisting of kestose (glucose-fructose-fructose or GF₂), nystose (GF₃), fructosyl-nystose (GF₄), GF₅, or GF_{5+m (where m=1 to 100)}, and combinations thereof.

16. The method of claim 10, that is in the form of a tablet, a powder or a liquid.

17. The method of claim 10, wherein the composition is administered to a mammalian subject in an amount from about 0.001 mg/kg to about 1 g/kg.

18. The method of claim 10, wherein the composition is administered to the mammalian subject daily.

19. The method of claim 10, wherein the composition is administered to the mammalian subject at least 3 days a week.
